(19) United States
(12) Patent Application Publication
(10) Pub. No.: US 2005/0112087 A1
(43) Pub. Date: May 26, 2005

(54) Pharmaceutical formulations for sustained drug delivery

(76) Inventors: Gary F. Musso, Hopkinton, MA (US); Nicholas Barker, Southborough, MA (US); Janet L. Wolfe, Newton, MA (US); Ming Ye, Acton, MA (US)

Correspondence Address: LAHIVE & COCKFIELD, LLP., 28 STATE STREET, BOSTON, MA, 02109, US

(21) Appl. No.: 10/835,717

(22) Filed: Apr. 29, 2004

Related U.S. Application Data
(60) Provisional application No. 60/466,388, filed on Apr. 29, 2003.

(51) Int. Cl. A61K 38/17; A61K 31/736; A61K 31/785

(52) U.S. Cl. 424/78.27; 514/12; 514/54

(57) ABSTRACT

The present invention provides pharmaceutical formulations comprising a solid ionic complex of a polypeptide having an isoelectric point lower than physiological pH and an anionic carrier molecule. The formulations of the invention are suitable as depot formulations for the sustained release of therapeutic polypeptides.

### RELATED APPLICATION

This application claims priority to U.S. Provisional Patent Application Ser. No.60/466388, filed on Apr. 29, 2003, the entire contents of which are incorporated herein by reference.

### BACKGROUND

A variety of diseases and clinical disorders are treated by the administration of a pharmaceutically active peptide.

In many instances, the therapeutic effectiveness of a pharmaceutically active peptide depends upon its continued presence in vivo over prolonged time periods. To achieve continuous delivery of the peptide in vivo, a sustained release or sustained delivery formulation is desirable, to avoid the need for repeated administrations. One approach for sustained drug delivery is by microencapsulation, in which the active ingredient is enclosed within a polymeric membrane to produce microparticles. Additional sustained delivery formulations for administering pharmaceutically active peptides in vivo continuously for prolonged time periods are needed.

### SUMMARY

The present invention provides pharmaceutical formulations comprising a solid ionic complex of a polypeptide having an isoelectric point lower than physiological pH and an anionic carrier molecule. The formulations of the invention are suitable as depot formulations for the sustained release of therapeutic polypeptides.

The polypeptide can be, for example, a monomeric or multimeric protein having a therapeutic activity. Preferred polypeptides can have a molecular weight of 100,000 daltons or less, 50,000 daltons or less, 40,000 daltons or less, 30,000 daltons or less, 20,000 daltons or less, 10,000 daltons or less, 5,000 daltons or less or 2,000 daltons or less. For example, the polypeptide can be composed of 2 or more, preferably five or more, amino acid residues. In one embodiment, the polypeptide comprises a single peptide chain composed of 1000 or fewer amino acid residues. In another embodiment, the polypeptide comprises a peptide chain composed of from about 5 to about 50 amino acid residues. The polypeptide can also comprise two or more peptide chains which are joined together covalently, for example, by disulfide bridges. Each of these chains can be composed of from about 5 to about 1000 amino acid residues, from about 5 to about 500 residues, from about 5 to about 300 residues or from about 5 to about 100 residues. Particular polypeptides which can be formulated as described herein include, but are not limited to, peptide hormones, enzymes useful for enzyme replacement therapy, non-naturally occurring peptides and protein fragments having useful therapeutic acitivity, cytokines, lymphokines and chemokines having isoelectric points below physiological pH.

Polypeptides which can be formulated according to the present invention include polypeptides having an isoelectric point which is below physiological pH. As used herein, the term “physiological pH” refers to a pH of 7.4. Preferably, the polypeptide has an isoelectric point less than about 7.0, less than about 6.5 or less than about 6.0. In preferred embodiments, the polypeptide has an isoelectric point which is between about 4.0 and about 7.0, more preferably between about 4.5 and about 6.5, and most preferably between about 5.0 and about 6.5. For example, the polypeptide can have a pI of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8 or 6.9.

Specific examples of polypeptides which can be formulated according to the present invention include insulin, growth hormone, erythropoietin, interferon-α, relaxin B-chain, granulocyte-monocyte colony stimulating factor, monocyte colony stimulating factor, granulocyte colony stimulating factor, epithelial growth factor, insulin-like growth factor II, angiotensin I, glucagon, calcitonin, interleukin-12α, interleukin-12β, interleukin-6, interleukin-15, interleukin-16, interleukin-18, adrenocorticotropic hormone, prolactin, stem cell factor, stem cell factor extracellular domain, factor VIIIa, bone morphogenic protein, prothrombin, lipotropin-β, lipotropin-γ, melanotropin-α, melanotropin-β, neurophysin-I, neurophysin-II, endothelin 1, endothelin-II, Von Willebrand's factor and Protein C.

Suitable peptides further include sequence variants and other analogues of the specific polypeptides set forth above having desirable therapeutic activity. For example, variants having structural modifications which result in an improved property, such as increase stability, bioavailability or therapeutic activity, or decreased side effect profile, are included. Such variants include sequence variants, in which one or more amino acid residues of the parent polypeptide have been replaced with another amino acid residue, such as a conservative substitution or a non-natural amino acid residue. The variant can also be a fragment of the parent polypeptide, resulting, for example, from the removal of one or more amino acid residues at the N— and/or C-terminus of the parent polypeptide.

Polypeptides which can be formulated as described herein further include synthetic polypeptides which include one or more non-naturally occurring amino acid residues, such as L-amino acid residues having non-natural side chains or D-amino acid residues. Suitable polypeptides can further include polypeptides which comprise one or more peptidomimetic units, for example, one or more dipeptide, tripeptide, or tetrapeptide mimetic units as known in the art.

Further, the invention provides, in at least one embodiment, a pharmaceutical formulation comprising a solid ionic complex of a polypeptide having an isoelectric point higher than the physiological pH and in ionic carrier molecule. In a specific exemplification of this embodiment, the polypeptide can be somatostatin, or a synthetic polypeptide analogue of somatostatin, e.g., octreotide.

Polypeptides which are suitable for use in the present invention can be identified using methods known in the art. The isoelectric point of a polypeptide can be determined experimentally, for example, via isoelectric focussing, in which a polypeptide migrates in a pH gradient under the influence of an applied electric field. At its isoelectric pH (“isoelectric point” or “pI”) the polypeptide has no net electric charge and stops moving. The isoelectric point of a polypeptide can also be estimated theoretically based on the amino acid sequence of the polypeptide. Such calculated isoelectric points, however, fail to account for post-translational modifications, such as glycosylation, and the effects of the local environment on the pKa of amino acid side chains, which can significantly alter the acidity of a functional group.

The anionic carrier macromolecule is preferably a linear or cross-linked polymer comprising monomers which bear a negative charge at physiological pH. In one embodiment, each of the monomeric units in the polymer comprises an acidic functional group or a salt thereof. In another embodiment, a fraction of the monomers within the polymer are functionalized with an acidic functional group. Preferably, the polymer comprises either anionic functional groups or cationic functional groups, although the polymer can comprise both cationic and anionic functional groups, so long as the proportion of these groups allows for the desired net anionic charge at physiological pH. Each of the cationic or anionic groups in the polymer can be the same or different, although in preferred embodiments they are the same.

In one embodiment, the polymer includes acidic or anionic functional groups, such as carboxylate, sulfonate, phosphonate, sulfate ester, phosphate ester, sulfamate or carbamate groups. Preferably the anionic groups are carboxylate groups.

The anionic carrier macromolecule is physiologically compatible and is, preferably, biodegradable or bioresorbable. Preferred anionic carrier macromolecules are suitable for administration via intraperitoneal, intramuscular or intravenous injection or inhalation. Suitable anionic polymers include anionic polysaccharides; anionic polyesters; anionic polyamides, for example, anionic peptides; and polyacrylates.

Examples of suitable anionic polymers include, but are not limited to, carboxymethylcellulose, poly(glutamic acid), poly(aspartic acid), poly(glutamic acid-co-glycine), poly(aspartic acid-co-glycine), poly(glutamic acid-co-alanine), poly(aspartic acid-co-alanine), starch glycolate, polygalacturonic acid, poly(acrylic acid) and alginic acid.

Preferred anionic polymers include anionic polysaccharides and anionic polypeptides. The anionic polymer can be linear or cross-linked. For example, the anionic polymer can be cross-linked to varying extents, for example, via ionic cross-linking or covalent cross-linking. In one embodiment, the anionic polymer bears a net anionic charge and is cross-linked by the addition of an amount of a cationic cross-linking polymer. The relative amounts of the two polymers can be varied to provide different degrees of cross-linking, but should be such that the combination retains a net ionic charge sufficient to bind a desired amount of the polypeptide. For example, an anionic polymer, such as carboxymethylcellulose, can be cross-linked with varying amounts of a cationic polymer, such as poly(lysine).

In another embodiment, the anionic polymer is covalently cross-linked. In a first example, an anionic polymer comprising carboxylate groups is covalently cross-linked as is known in the art by reacting a fraction of the carbosylate groups, or activated derivatives thereof, with a suitable cross-linking reagent such as a dialcohol, an aminoalcohol or a diamine, under conditions suitable for forming ester and/or amide linkages. In this case, the ionic polymer will comprise carboxylate groups and ester/amide groups, with the ester/amide groups on one polymer strand linked to ester/amide groups on another polymer strand by bridging groups derived from the dialcohol, amino alcohol or diamine used. Preferably, the dialcohol, amino alcohol or diamine is pharmaceutically acceptable.

The solid ionic complex an have a range of compositions. For example, the complex can comprise from about 2% polypeptide to about 95% polypeptide. The complex can comprise from about 98% anionic macromolecule to about 5% anionic macromolecule. Preferably, the solid ionic complex comprises 10% or greater, 20% or greater or 30% or greater polypeptide. More preferably, the solid ionic complex comprises 40% or greater or 50% or greater polypeptide. Preferably, the solid ionic complex comprises 90% or less; 80% or less; or 70% or less anionic macromolecule. More preferably, the solid ionic complex comprises 60% or less or 50% or less anionic macromolecule. All percentages disclosed herein are weight/weight unless otherwise indicated.

The ratio (weight/weight) of the polypeptide to the ionic macromolecule in the solid ionic complex ofthe invention is preferably about 10, 9, 8, 7, 6, 5, 4, 3, 2, 1, 0.75, 0.5, 0.25, or 0.1. Preferably the ratio of the polypeptide to the ionic macromolecule is about 0.5, 0.75, 1 or greater.

In one embodiment, the solid ionic complex consists essentially of the anionic macromolecule and the polypeptide. Typically, such a solid ionic complex will be hydrated and the mass of the complex will include some amount of water. The degree of hydration can be determined by subjecting the complex to dehydrating conditions, preferably conditions under which the polypeptide and the anionic macromolecule are stable, and determining the resulting weight decrease.

In another embodiment, the solid ionic complex comprises a polypeptide having an isoelectric point below physiological pH, the anionic carrier macromolecule and one or more additional substances. Suitable additional substances include a second pharmaceutically active compound, which, preferably, has a net positive change at physiological pH. The additional substance or substances can also include one or more pharmaceutically acceptable excipients or other agents which modulate the properties of the complex, such as solubility.

The solid ionic complex is, preferably, substantially insoluble in aqueous solvent at physiological pH. The term “substantially insoluble”is used herein to refer to a material that has limited solubility under a given set of conditions. It is to be understood that a substantially insoluble material can have finite solubility, but generally is soluble to an extent providing a concentration of pharmaceutically active agent no greater than 10 mM, 1 mM, 100 μM, 10 μM or 1 μM. For a given polypeptide, the anionic carrier macromolecule and additional excipients, if any, can be selected to optimize the properties of the solid ionic complex with respect to aqueous solubility and/or polypeptide content, among others. For example, cross-linking is expected to reduce the solubility of the resulting complexes and can be accomplished using methods known in the art, such as covalent cross-linking or ionic cross-linking, as discussed above.

The solubility of the solid ionic complex can also be modulated by including in the complex an excipient such as one or more di- or trivalent metal cations, such as Al³⁺, Ca²⁺, Fe²⁺, Fe³⁺ or Mg²⁺. The metal cation can be added in varying amounts as required to obtain the desired solubility. For example, the metal cation(s) can be added in an amount required to neutralize from 0.01% to 50% of the anionic groups on the anionic carrier macromolecule. Preferably, the metal cation is added in an amount required to neutralize from 0.01% up to 2%, 5%, 7%, 10%, 12%, 15%, 17% or 20% of the anionic groups on the anionic carrier macromolecule. One of skill in the art can readily determine a combination of excipients, cross-linking agents and extent of cross-linking to provide a complex having the desired solubility.

The present invention further includes pharmaceutical compositions comprising a solid ionic complex of a pharmaceutically active compound and an ionic carrier molecule and a pharmaceutically acceptable carrier. For example, the solid ionic complex can be suspended in a vehicle suitable for injection, water for injection, a buffered aqueous solution, or an oil-based vehicle.

The pharmaceutical composition can also include the solid ionic complex and a carrier suitable for administration via inhalation. Particular compositions suitable for inhalation include dry powders, liquid solutions or suspensions suitable for nebulization, and propellant formulations suitable for use in metered dose inhalers (MDIs). Suitable carriers for inhalation include dry bulking powders, such as sucrose, lactose, trehalose, human serum albumin (HAS), and glycine. Other suitable dry bulking powders include cellobiose, dextrans, maltotriose, pectin, sodium citrate, sodium ascorbate and mannitol. The solid ionic complex can also be suspended in a suitable aerosol propellant, such as a chlorofluorocarbon (CFC) or a hydrofluorocarbon (HFC). Suitable CFCs include trichloromonofluoromethane (propellant 11), dichlorotetrafluoromethane (propellant 114), and dichlorodifluoromethane (propellant 12). Suitable HFCs include tetrafluoroethane (HFC-134a) and heptafluoropropane (HFC-227). Preferably, for incorporation into the aerosol propellant, the solid ionic complex of the present invention can be processed into respirable particles. The particles are then suspended in the propellant, and, optionally, coated with a surfactant to enhance their dispersion. Suitable surfactants include oleic acid, sorbitan trioleate, and various long chain diglycerides and phospholipids. The inhalable compositions of the invention can be administered using a conventional dry powder inhaler, nebulizer or metered dose inhaler.

The pharmaceutical composition can also be suitable for oral administration. For example, the pharmaceutical composition can include the solid ionic complex and a coating or carrier which protects the complex from the acidic environment of the stomach. The solid ionic complex and any excipients can be, for example, covered by an enteric coating.

Preparation of Compositions

The present invention also relates to a method of preparing a solid ionic complex comprising an ionic macromolecule and a pharmaceutically active compound. The solid ionic complex of the invention is prepared by combining the pharmaceutically active compound and the carrier macromolecule under conditions such that a water-insoluble complex of the pharmaceutically active compound and the ionic carrier macromolecule forms. In one embodiment, the method comprises the steps of (1) providing a polypeptide having an isoelectric point below physiological pH and an anionic carrier macromolecule; and (2) combining the polypeptide and the anionic carrier macromolecule under conditions such that a water-insoluble complex of the pharmaceutically active compound and the carrier macromolecule forms. Preferably, the polypeptide and the anionic macromolecule are combined in an aqueous solvent at a pH below the isoelectric point of the polypeptide. For example, the polypeptide and the anionic carrier macromolecule can be combined in solution in an aqueous buffer at a pH below the isoelectric point of the polypeptide. In one embodiment, the pH is no more than 2 pH units below the isoelectric point of the polypeptide; preferably the pH is no more than one pH unit below the isoelectric point of the polypeptide.

The anionic macromolecule can be combined with the polypeptide in a variety of ways. For example, a solution of the anionic macromolecule can be mixed with a solution of the polypeptide under conditions suitable for precipitation of the solid ionic complex. The two solutions can include the same solvent or different solvents. Preferably, if the solvents are different, they are miscible. Alternately, the anionic macromolecule can be added as a solid to a solution of the polypeptide or the polypeptide can be added to a solution of the ionic macromolecule.

In another embodiment, the ionic macromolecule and the polypeptide are added to a solvent in which neither is substantially soluble, but in which a by-product of the complexation, or ion-exchange process, is soluble. For example, a polypeptide which forms a water-insoluble hydrochloride salt can be added to an aqueous suspension of the sodium salt of an anionic macromolecule. The resulting suspension can be agitated for a sufficient period of time for formation of the desired solid ionic complex. In this case, the ion exchange process resulting in the desired solid ionic complex is driven, at least in part, by the solubility of the sodium chloride product.

Once the solid ionic complex precipitates, the precipitate can be removed from the solution by means known in the art, such as filtration (e.g., through a 0.45 micron nylon membrane), centrifugation and the like. The recovered paste than can be dried (e.g., in vacuo or in a 70° C. oven or a vacuum oven), and the solid can be milled or pulverized to a powder by means known in the art (e.g., hammer or gore milling, or grinding in mortar and pestle). Following milling or pulverizing, the powder can be sieved through a screen (preferably a 90 micron screen) to obtain a uniform distribution of particles. Moreover, the recovered paste can be frozen and lyophilized to dryness. The powder form of the complex can be dispersed in a carrier solution to form a liquid suspension or semi-solid dispersion suitable for injection. Accordingly, in various embodiments, a pharmaceutical formulation of the invention is a dry solid, a liquid suspension or a semi-solid dispersion. Examples of liquid carriers suitable for use in liquid suspensions include saline solutions, glycerin solutions, lecithin solutions and oils suitable for injection.

In another embodiment, the pharmaceutical formulation of the invention is a sterile formulation. For example, following formation of the water-insoluble complex, the complex can be sterilized, optimally by gamma irradiation or electron beam sterilization. Accordingly, the method of the invention for preparing a pharmaceutical formulation described above can further comprise sterilizing the water-insoluble complex by gamma irradiation or electron beam irradiation. Preferably, the formulation is sterilized by gamma irradiation using a gamma irradiation dose of at least 15 Kgy. In other embodiments, the formulation is sterilized by gamma irradiation using a gamma irradiation dose of at least 19 KGy or at least 24 Kgy. Alternatively, to prepare a sterile pharmaceutical formulation, the water-insoluble complex can be isolated using conventional sterile techniques (e.g., using sterile starting materials and carrying out the production process aseptically). Accordingly, in another embodiment of the method for preparing a pharmaceutical formulation described above, the water-insoluble complex is formed using aseptic procedures.

Use of Compositions

In another embodiment, the present invention relates to a method of administering a polypeptide to a subject, where the polypeptide has an isoelectric point which is lower than physiological pH. The method comprises the steps of (1) providing a pharmaceutical composition comprising a solid ionic complex comprising the polypeptide and an anionic carrier macromolecule and (2) contacting the body of the subject with the pharmaceutical composition. The body of the subject can be contacted with the pharmaceutical composition by a variety of methods. For example, the pharmaceutical composition can be injected into the subject's body. The injection can be, for example, an intramuscular, intravenous, intraperitoneal or subcutaneous injection. The subject can also be caused to inhale or swallow the pharmaceutical composition. The subject's eye or eyes can also be contacted with the pharmaceutical composition.

The invention further relates to a method of treating a subject suffering from a medical condition for which a polypeptide having an isoelectric point below physiological pH is indicated. The method comprises the steps of (1) providing a pharmaceutical composition comprising a solid ionic complex of the polypeptide and an anionic carrier macromolecule; and (2) administering the pharmaceutical composition to the subject.

The subject can be an animal in need of treatment for which the pharmaceutically ctive compound is indicated, and is preferably a mammal, such as a canine, feline, bovine, equine, ovine or porcine animal or a primate, such as a monkey, an ape or a human. More preferably, the subject is a human. The subject can be an individual diagnosed with, or suspected of having, the medical condition, or an individual at risk of developing the medical condition.

The term “medical condition”, as used herein, is a disease or disorder which is susceptible to medical treatment. The subject is in need of treatment for a medical condition if modification or prevention of the condition is desirable, or if the subject would benefit from alleviation of the symptoms of the condition. As intended herein, a polypeptide is “indicated” for a medical condition if it provides therapeutic benefit to an individual having the medical condition or is of use in prevention (prophylaxis) of the medical condition.

Devices which can be used to administer the pharmaceutical compositions of the invention are also contemplated. One suitable example of such a device is a syringe which houses a pharmaceutical composition comprising a solid ionic complex comprising the polypeptide and an anionic carrier macromolecule, where the complex is suspended in a vehicle suitable for injection. Another suitable example is an inhalation device which houses a pharmaceutical composition comprising a solid ionic complex comprising the polypeptide and an anionic carrier macromolecule and a pharmaceutically acceptable carrier suitable for inhalation. The inhalation device can be, for example, a dry powder inhaler, a nebulizer or a metered dose inhaler.

Exemplification

Insulin Depot Formation and Characterization

Materials

Bovine insulin was obtained from Sigma Chemical Company (catalog no. I8405). Carboxymethylcellulose sodium (Low Viscosity, USP) was obtained from Spectrum Laboratory Products (Catalog no. CA 193; degree of substitution 0.84)

Preparation of Bovine Insulin-Carboxymethylcellulose Complex

Bovine insulin (756 mg) was dissolved in a minimal amount of 50% acetic acid in water and sufficient 5% acetic acid in water was added to obtain an insulin concentration of approximately 10 mg/mL. Sufficient 1% sodium hydroxide solution was then added to bring the pH to 3.9, resulting in an insulin concentration of 5.8 mg/mL. A 0.5% (weight/weight) solution of carboxymethylcellulose in water was prepared and filtered. 16 mL of the 0.5% CMC solution was added to the insulin solution with stirring and a white precipitate appeared immediately. After stirring for an additional hour, the precipitate was isolated by filtration and washed with water. An additional 100 mL water was added to the supernatant, causing the formation of more precipitate, which was isolated by centrifugation and washed with water. The wet solids were combined and dried in vacuo to yield 779 mg of a freely flowing white powder.

Analysis of the powder revealed the following composition (weight/weight): insulin 86.64%, CMC 7.50%; water 1.50%.

The solubility of the powder in a variety of media was determined and is shown in the table below

| Solvent | Solubility (mg complex/ML) |
|---|---|
| Water | 0.016 (0.014) |
| Ethanol | 0.012 (0.010) |
| 5% Dextrose | 0.018 (0.016) |
| Saline | 0.052 (0.045) |
| 0.33 M NaCl | 0.329 (0.285) |
| 5% Acetic Acid | 7.138 (6.182) |
| Organic Solvent | ≦0.01 mg/mL |
| (excluding DMSO) | |

1. A solid ionic complex comprising an anionic carrier macromolecule and a polypeptide having an isoelectric point less than about 7.4.

2. The solid ionic complex of claim 1 wherein the polypeptide has an isoelectric point less than about 7.0.

3. The solid ionic complex of claim 2 wherein the polypeptide has an isoelectric point less than about 6.5.

4. The solid ionic complex of claim 3 wherein the polypeptide has an isoelectric point less than about 6.0

5. The solid ionic complex of claim 4 wherein the polypeptide has an isoelectric point less than about 5.5.

6. The solid ionic complex of claim 5 wherein the polypeptide has an isoelectric point less than about 5.0.

7. The solid ionic complex of claim 1 wherein the polypeptide has an isoelectric point between about 4.5 and about 7.0.

8. The solid ionic complex of claim 7 wherein the polypeptide has an isoelectric point between about 5.0 and about 6.5.

9. The solid ionic complex of claim 1 wherein the anionic carrier macromolecule is a polypeptide or a polysaccharide.

10. The solid ionic complex of claim 9 wherein the anionic carrier macromolecule is selected from the group consisting of carboxymethylcellulose, poly(glutamic acid), poly(aspartic acid), poly(glutamic acid-co-glycine), poly(aspartic acid-co-glycine), poly(glutamic acid-co-alanine), poly(aspartic acid-co-alanine), starch glycolate, polygalacturonic acid, poly(acrylic acid) and alginic acid.

11. The solid ionic complex of claim 10 wherein the anionic carrier macromolecule is selected from the group consisting of poly(glutamic acid) and poly(aspartic acid).

12. The solid ionic complex of claim 10 wherein the anionic carrier macromolecule is carboxymethylcellulose.

13. The solid ionic complex of any of claims 1 to 12, wherein the polypeptide is selected from the group consisting of peptide hormones, enzymes useful for enzyme replacement therapy, non-naturally occurring peptides and protein fragments having useful therapeutic acitivity, cytokines, lymphokines and chemokines having isoelectric points below physiological pH.

14. The solid ionic complex of any of claims 1 to 12 wherein the polypeptide is selected from the group consisting of insulin, growth hormone, erythropoietin, interferon-α, relaxin B-chain, granulocyte-monocyte colony stimulating factor, monocyte colony stimulating factor, granulocyte colony stimulating factor, epithelial growth factor, insulin-like growth factor II, angiotensin I, glucagon, calcitonin, interleukin-12α, interleukin-12β, interleukin-6, interleukin-15, interleukin-16, interleukin-18, adrenocorticotropic hormone, prolactin, stem cell factor, stem cell factor extracellular domain, factor VIIa, bone morphogenic protein, prothrombin, lipotropin-β, lipotropin-γ, melanotropin-α, melanotropin-β, neurophysin-I, neurophysin-II, endothelin 1, endothelin-II. Von Willebrand's factor, Protein C.

15. A pharmaceutical composition comprising the solid ionic complex of any of claims 1 to 12 and a pharmaceutically acceptable carrier.

16. The pharmaceutical composition of claim 15 wherein the pharmaceutically acceptable carrier is a liquid suitable for injection.

17. A method of administering a polypeptide to a subject, said polypeptide having an isoelectric point below physiological pH, comprising the steps of:
(a) providing a pharmaceutical composition comprising a solid ionic complex of any one of claims 1-12; and
(b) contacting the subject's body with the pharmaceutical composition by a method selected from the group consisting of
(i) injecting the pharmaceutical composition into the subject's body;
(ii) causing the subject to inhale the pharmaceutical composition
(iii) causing the subject to swallow the pharmaceutical composition; and
(iv) contacting the eyes of the subject with the pharmaceutical composition.

18. A method of treating a subject having a medical condition for which a polypeptide having an isoelectric point below physiological pH is indicated, said method comprising the step of administering to the subject a therapeutically effective amount of a pharmaceutical composition comprising a solid ionic complex of any one of claims 1 to 12.
